(19) United States
(12) Patent Application Publication
(10) Pub. No.: US 2020/0376188 A1
(43) Pub. Date: Dec. 3, 2020

(54) System for Integrating an Antibacterial-Element Receptacle into a Multi-Function Cap

(71) Applicant: Silcor Biomed, LLC., Deerfield Beach, FL (US)

(72) Inventors: Silvestre Cordoba, Deerfield Beach, FL (US); Frank A. Scarfone, Miramar, FL (US)

(21) Appl. No.: 16/997,773

(22) Filed: Aug. 19, 2020

Related U.S. Application Data
(63) Continuation-in-part of application No. 15/925,648, filed on Mar. 19, 2018, now Pat. No. 10,780,256.
(60) Provisional application No. 62/472,875, filed on Mar. 17, 2017.

(51) Int. Cl. A61M 5/00; A61M 5/32; A61M 5/20

(52) U.S. Cl. CPC A61M 5/001; A61M 2005/2073; A61M 5/20; A61M 5/3204

(57) ABSTRACT

A system for integrating an antibacterial-element receptacle into a multi-function cap facilitates the pre-injection treatment, the post-injection treatment, and prevents the accidental delivery of the injection. The system includes a multi-function cap assembly, an autoinjector body, and an antiseptic-soaked dressing. The multi-function cap assembly encloses the needle of the autoinjector and prevents the accidental trigger of the injection mechanism. The autoinjector body refers to the body of any conventional autoinjector. The antiseptic-soaked dressing is applied to the patient's skin prior to the delivery of the injection to disinfect and sterilize the injection area. The antiseptic-soaked dressing is housed in the multi-function cap assembly, between a receptacle and a removable cover. The removable cover is releasably mounted to the receptacle to allow access to the antiseptic-soaked dressing. The apparatus further includes an expiration timer and a locking mechanism. The locking mechanism, with the timer, secures multi-function cap assembly with the body.

The current application is a continuation-in-part (CIP) application of a U.S. non-provisional application Ser. No. 15/925,648 filed on Mar. 19, 2018. The U.S. non-provisional application Ser. No. 15/925,648 claims a priority to a U.S. provisional application Ser. No. 62/472,875 filed on Mar. 17, 2017.

### FIELD OF THE INVENTION

The present invention generally relates to a system for attaching an antiseptic element receptacle into a multi-function cap. More specifically, the antiseptic element receptacle facilitates the pre-treating of the injection area, the administration of the injection, and in some embodiments, the post-injection treatment process.

### BACKGROUND OF THE INVENTION

There are many different forms of autoinjectors for self-administering medication in their liquid forms. Although these autoinjectors are designed to be used by patients, they can also be delivered by trained medical personnel. An autoinjector is a medical device designed to deliver a single dose of a (typically life-saving) drug. By design, most autoinjectors are spring loaded syringes that are easy to operate and can be operated by untrained personnel or by the patients themselves.

Before administering the injection via an autoinjector, it is important for the injection site, where the needle is to subcutaneously penetrate the patient's skin, to be disinfected and free of germs that can cause infections and post-injection complications. Typically, the administrator swabs the injection site with a dressing infused with an antiseptic, antibacterial solution. The dressing is typically a cloth swab pre-soaked with the antiseptic or may be used in conjunction with a bottle of an antiseptic and that is poured onto the swab just before the pre-treatment of the injection area. One of the issues with home autoinjector usage is that it is not always convenient to have the swabs or other types of cleansing items available when it is time to administer a drug via injection. The swabs themselves are normally sold in large, bulky quantities that are difficult to carry and transport. In most cases, swabs are stored in medical kits carried by medical personnel. Further, it is also possible for the patient to be out of swabs or the antiseptic, antibacterial solution when the trying to deliver the injection.

Thus, there exists a need for a system to integrate swabs and antiseptic, antibacterial solutions, into a compact, easy-to-carry package that can be mounted to the autoinjector itself. More specifically, the present invention utilizes a multi-function cap capable of holding materials for both the pre-injection treatment process and the post-injection treatment process.

### SUMMARY OF THE INVENTION

The present invention is a system for integrating an antibacterial element receptacle into a multi-function cap. The system includes a multi-function cap assembly coupled to a needle-end of the autoinjector housing. The multi-function cap assembly contains an antiseptic-soaked dressing. The multi-function cap assembly includes a removable cover and a receptacle for enclosing the antiseptic-soaked dressing. In one possible embodiment, the antiseptic-soaked dressing may be positioned into an opening of the receptacle, allowing the antiseptic-soaked dressing to be utilized while the multi-function cap assembly is attached to the body. In another possible embodiment of the present invention, the multi-function cap assembly contains a storage compartment for storing post-injection treatment materials. Further, in this embodiment, the antiseptic-soaked dressing is mounted into an opening of the removable cover, thereby requiring the administrator to remove the multi-function cap assembly from the body for the pre-injection treatment process. In yet another embodiment, the multi-function cap assembly integrates an expiration timer with a locking mechanism, designed as an automated safety system. The expiration timer is programmed to actuate the locking mechanism once the expiration date of the medicine passes, thus preventing the use of the expired medication. The expiration timer/locking mechanism shall have a digital readout clock with options to display days to expire or the actual date of expiration. Additionally, a flashing LED warning light is incorporated to alert the user.

### BRIEF DESCRIPTION OF THE DRAWINGS

FIG. 1 is a front perspective view of the present invention, wherein the antiseptic-soaked dressing is attached to the receptacle.

FIG. 2 is a front perspective view illustrating the multi-function cap assembly with the removable cover detached from the receptacle.

FIG. 3 is a top exploded view of the present invention.

FIG. 4 is a cross sectional view taken along line 4-4 in FIG. 3 illustrating the cover fastener and the coupling interface.

FIG. 5 is a front perspective view of an alternate embodiment, wherein the antiseptic-soaked dressing is attached to the removable cover.

FIG. 6 is a front perspective view of the alternate embodiment with the removable cover detached from the receptacle.

FIG. 7 is an exploded view of the alternate embodiment showing the cover lid detached from the storage compartment.

FIG. 8 is a rear perspective exploded view of alternate embodiment illustrating the cover fastener.

FIG. 9 is rear perspective view of the present invention illustrating the operation of the locking mechanism.

FIG. 10 is a front perspective view of an alternate embodiment, wherein a coupling interface is positioned opposite a needle-shield remover about the body.

FIG. 11 is a top exploded view of the alternate embodiment present invention with the coupling interface positioned opposite the needle-shield remover about the body.

### DETAILED DESCRIPTION OF THE INVENTION

All illustrations of the drawings are for the purpose of describing selected versions of the present invention and are not intended to limit the scope of the present invention.

The present invention is a system for integrating an antibacterial element receptacle into a multi-function cap. By mating the antibacterial element to the multi-function cap, the present invention facilitates the pre-treating of the injection area, the administration of the injection, and in some embodiments, the post-injection treatment process.

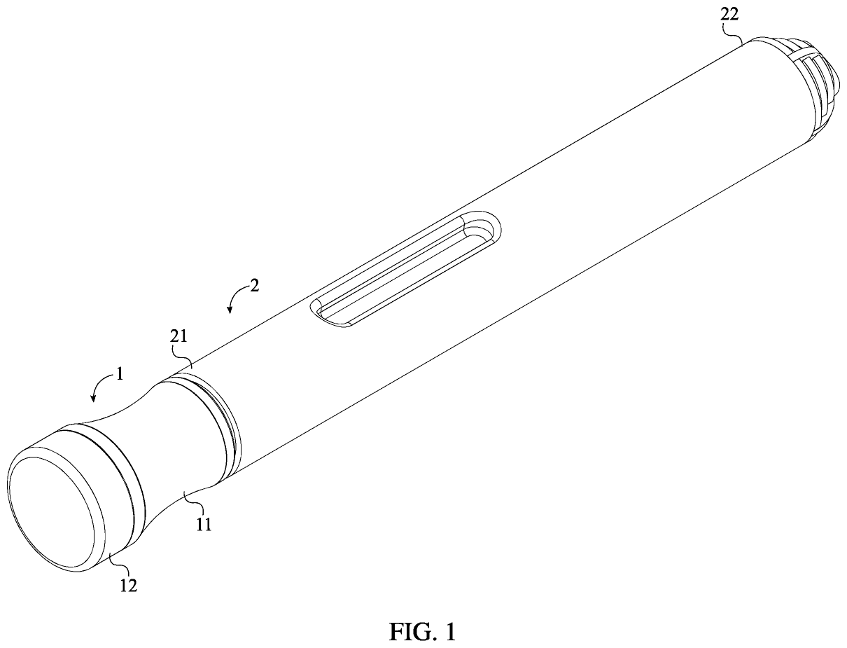

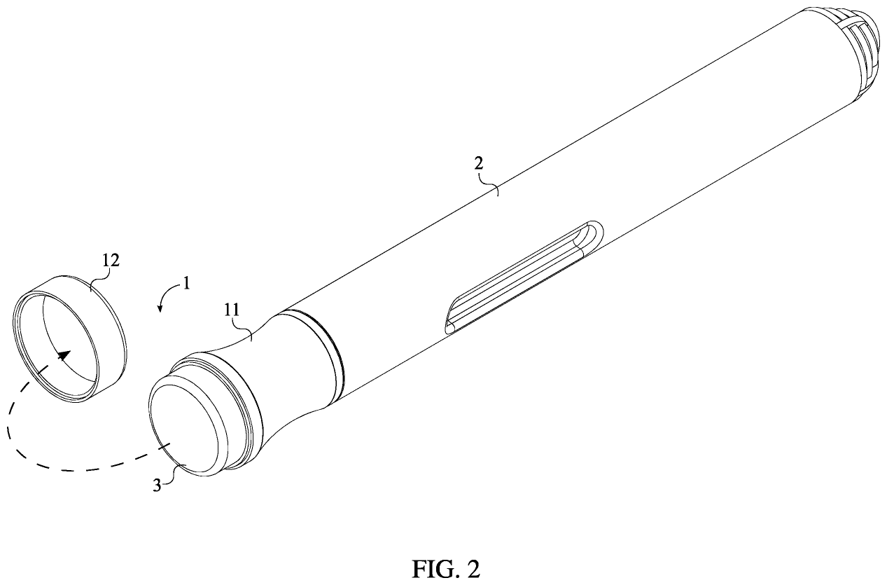

In reference to FIG. 1 and FIG. 2, the preferred embodiment of the present invention comprises a multi-function cap assembly 1, a body 2, and an antiseptic-soaked dressing 3. The multi-function cap assembly 1 covers the needle of the autoinjector until the injection needs to be administered to a patient. The multi-function cap assembly 1 of the present invention, in addition to covering the needle, houses materials that are required for the pre-injection treatment. The multi-function cap assembly 1 must be made of materials that can be easily sterilized such as carbon steel, stainless steel, aluminum, Titanium, other metals or alloys, composites, ceramics, polymeric materials such as polycarbonates. Preferably, the multi-function cap assembly 1 is designed to be retrofitted onto an existing autoinjector. As such, the body 2 refers to the body of any conventional autoinjector known in the public domain. Alternately, the multi-function cap assembly 1 may be retrofitted onto a custom designed autoinjector. Finally, the antiseptic-soaked dressing 3 is wiped onto the skin of the patient, where the injection is to be administered.

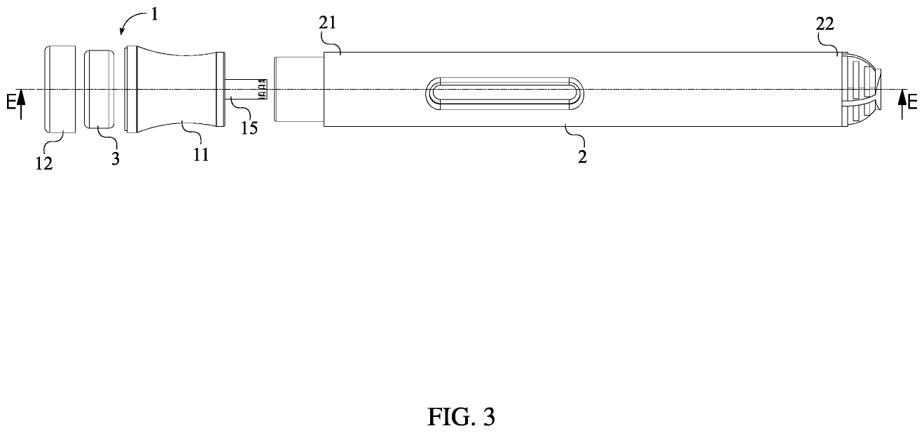

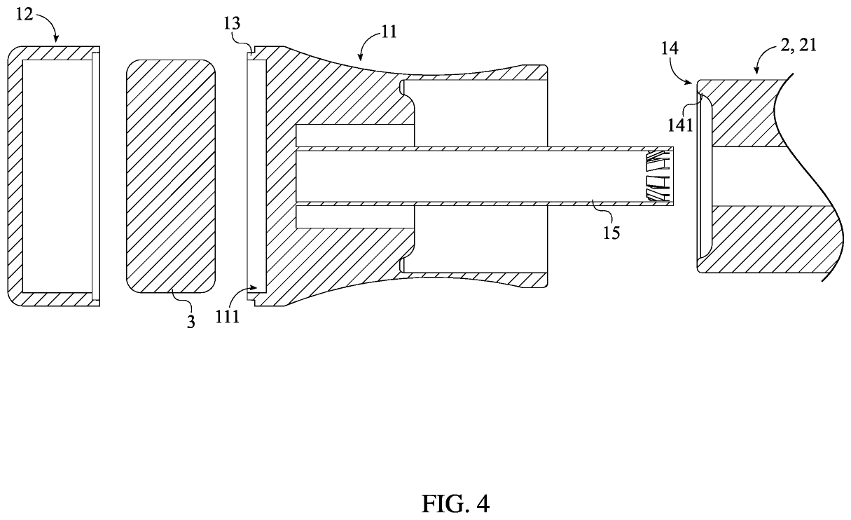

Referring to FIG. 3 and FIG. 4, preferably, the antiseptic-soaked dressing 3 is impregnated with an antiseptic-antibacterial solution that disinfects and sanitizes the patient's skin. As the name implies, the antiseptic-soaked dressing 3 is made of absorbent foam or cloth that absorbs and retains the antibacterial-antiseptic solution until the administration of the injection. Unlike conventional autoinjectors which require a separate medical kit for holding the antiseptic-soaked dressing 3, the present invention uses the multi-function cap assembly 1 to house the antiseptic-soaked dressing 3. As such, the multi-function cap assembly 1 comprises a receptacle 11, a removable cover 12, a cover fastener 13, and a coupling interface 14. The receptacle 11 is preferably a hollow cylindrical structure that houses the antiseptic-soaked dressing 3. The removable cover 12 is similarly a hollow cylindrical structure that seals the antiseptic-soaked dressing 3 within the receptacle 11, thereby preventing the antiseptic-soaked dressing 3 from drying out. The cover fastener 13 fastens the removable cover 12 to the receptacle 11. Similarly, the coupling interface 14 couples the receptacle 11 to the body 2, thereby coupling the multi-function cap assembly 1 to the body 2 as well. The antiseptic-soaked dressing 3 is mounted within the multi-function cap assembly 1. Placing the antiseptic-soaked dressing 3 in the multi-function cap assembly 1 for pre-injection treatment obviates the need to carry pre-injection treatment materials in a separate container.

Referring more specifically to FIG. 4, the removable cover 12 is attached across an opening 111 of the receptacle 11 by the cover fastener 13. Similarly, the coupling interface 14 is connected adjacent to the receptacle 11, opposite the removable cover 12. More specifically, the body 2 and the removable cover 12 attach at opposite ends of the multi-function cap assembly 1. As such, the antiseptic-soaked dressing 3 is positioned between the receptacle 11 and the removable cover 12. To connect the multi-function cap assembly 1 to the body 2, the coupling interface 14 is engaged to the body 2. A pull tab that is preferably mounted to the rim of the removable cover 12 allows the removable cover 12 to easily disengage the receptacle 11, thereby exposing the antiseptic-soaked dressing 3. The administrator may then rub the antiseptic-soaked dressing 3 against the patient's skin, thereby treating the injection area. Once the injection area is treated, the administrator may remove the receptacle 11 and deliver the injection.

In the preferred embodiment of the present invention, the coupling interface 14 is a slip-fit fastener 141 that selectively fastens the multi-function cap assembly 1 to the autoinjector. The slip-fit fastener 141 can establish a secure connection between the receptacle 11 and a variety of different autoinjectors currently in the market. Alternately, the coupling interface 14 may utilize snaps, screws, magnets, or any number of fastening mechanisms capable of securely connecting with the body 2. The slip-fit fastener 141 is a cylindrical extrusion fabricated to fine tolerances. The slip-fit fastener 141 slides into a cylindrical slot made into the body 2 that is dimensioned slightly thicker than the slip-fit fastener 141. The cylindrical slot is positioned adjacent to the needle-end of the body 2, thereby allowing the multi-function cap assembly 1 to cover the needle until the time of the delivery.

Referring more specifically to FIG. 2 and FIG. 4, in the preferred embodiment of the present invention, the antiseptic-soaked dressing 3 is connected into the receptacle 11. As a result, the administrator can apply the antiseptic-soaked dressing 3 to the injection area without removing the receptacle 11 from the body 2. In addition, the administrator can leverage the body 2 to more accurately apply the antiseptic-soaked dressing 3 to the desired injection area. Preferably, the antiseptic-soaked dressing 3 is attached or fastened into the opening 111 of the receptacle 11. More specifically, the antiseptic-soaked dressing 3 may be attached or coupled to the opening 111 of the receptacle 11 via a friction fit, snap fit adhesive, fastener, or another suitable means that is within the spirit and scope of the present invention. As the antiseptic-soaked dressing 3 is longer than the depth of the cavity, the antiseptic-soaked dressing 3 traverses out of the opening 111 of the receptacle 11. As such, the removable cover 12 is used to enclose the exposed end of the antiseptic-soaked dressing 3.

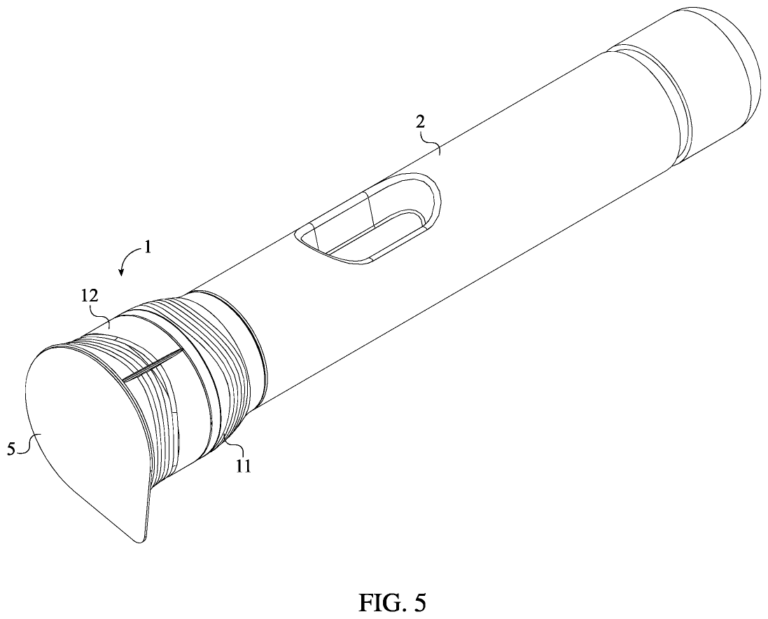

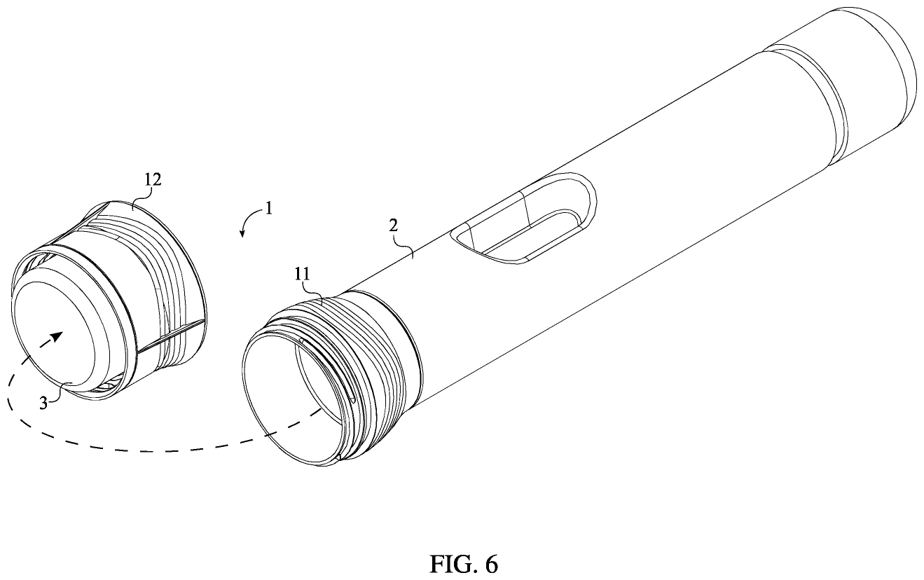

Referring to FIG. 5 and FIG. 6, in an alternate embodiment of the present invention, the antiseptic-soaked dressing 3 is connected to the removable cover 12. More specifically, the antiseptic-soaked dressing 3 is attached or fastened onto an opening of the removable cover 12. The antiseptic-soaked dressing 3 traverses into the opening 111 of the receptacle 11. Thus, similarly allowing the antiseptic-soaked dressing 3 to be positioned in between the receptacle 11 and the removable cover 12. In this embodiment, the administrator must separate the antiseptic-soaked dressing 3 from the body 2 to treat the injection area. This prevents accidentally activating the injection mechanism of the autoinjector while treating the injection area.

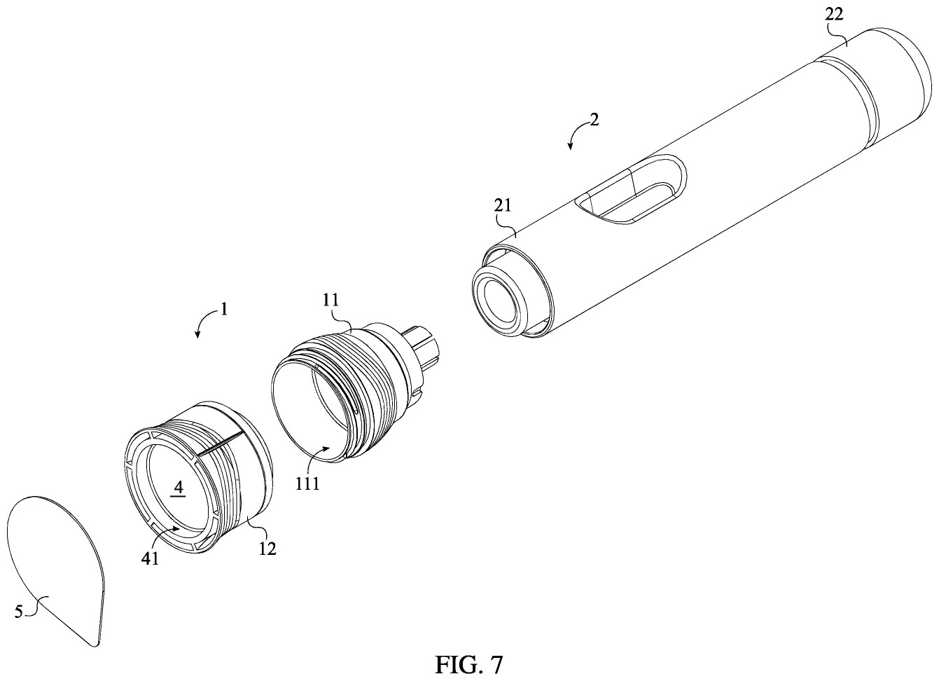

Referring to FIG. 7, as mentioned previously, the present invention is also a system for facilitating the post-injection treatment process. As such, a storage compartment 4 is provided to house post-injection treatment materials. The storage compartment 4 is secured with a cover lid 5. Preferably, the storage compartment 4 is integrated into the removable cover 12, opposite to the receptacle 11. Further, the cover lid 5 is attached across an opening 41 of the storage compartment 4. The opening 41 preferably faces away from the body 2 in the engaged position. As such, the storage compartment 4 can be accessed without having to disengage the removable cover 12 from the receptacle 11. Post-injection treatment materials may refer to a gauze and a bandage for covering the injection area. The cover lid 5 is designed to securely enclose the storage compartment 4 until it is time to deliver the injection. Preferably, the cover lid 5 is a die-cut sheet of aluminum that is heat sealed onto the removable cover 12. Alternately, the cover lid 5 can be any type of plastic or paper closure. As such, the cover lid 5 is reinforced against forward and side impacts, as those that occur from being dropped on the floor or knocked against hard objects. However, the heat seal is weak enough to allow the administrator to easily peel the cover lid 5 off the removable cover 12 to access the gauze and the bandage. Once the injection is successfully delivered, the administrator uses the gauze and bandages in the storage compartment 4 to dress the injection area. More specifically, the administrator may position the gauze over the injection area such that the gauze absorbs blood escaping from the hole left by the needle of the body 2. Next, the administrator may attach the bandage over the gauze to secure the gauze to the patient's body.

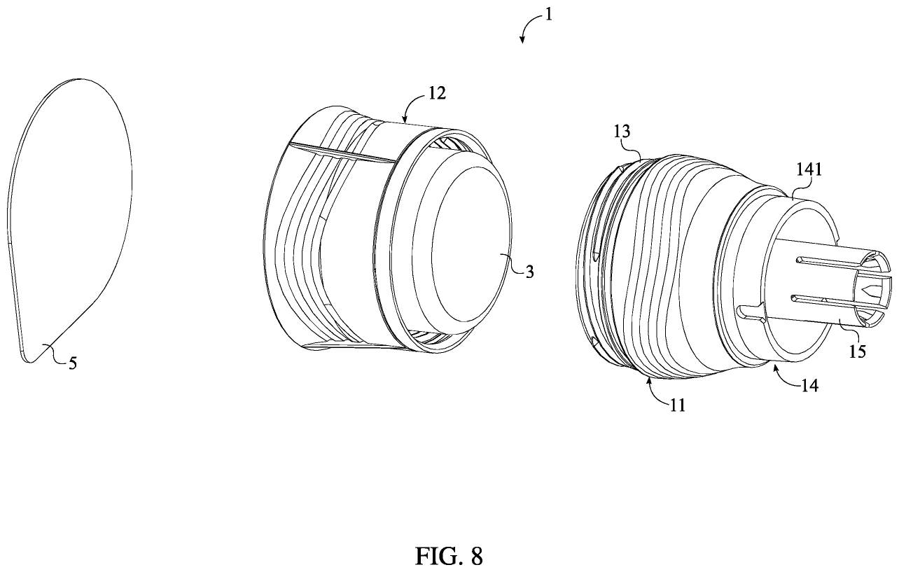

Referring to FIG. 8, when the antiseptic-soaked dressing 3 is connected to the removable cover 12, the removable cover 12 is threadably engaged to the receptacle 11. More specifically, the cover fastener 13 comprises external threads and internal threads. The external threads are terminally connected to the receptacle 11, whereas the internal threads are terminally connected to the removable cover 12. Accordingly, the removable cover 12 can be screwed onto the receptacle 11. Alternately, when the antiseptic-soaked dressing 3 is mounted onto the receptacle 11, the cover fastener 13 may be snap fasteners, slip-fit slots, and/or magnetic fasteners.

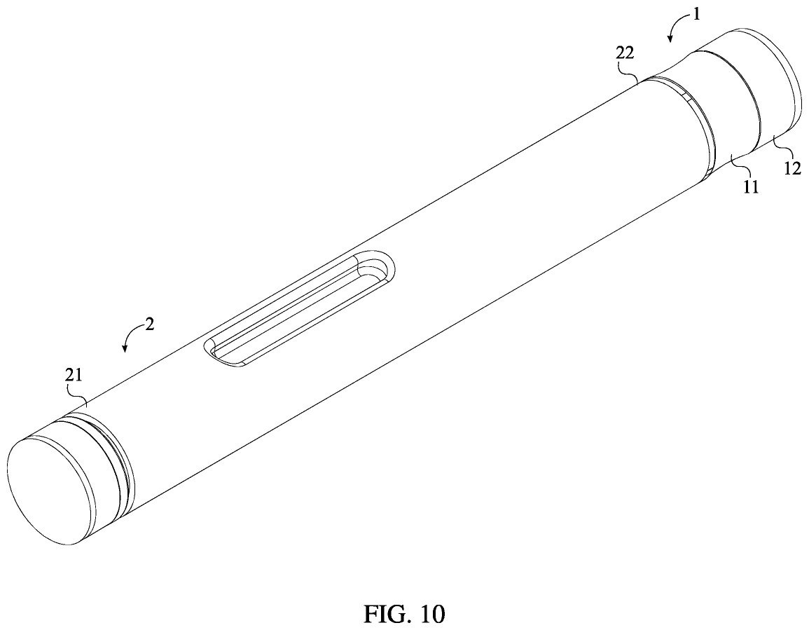

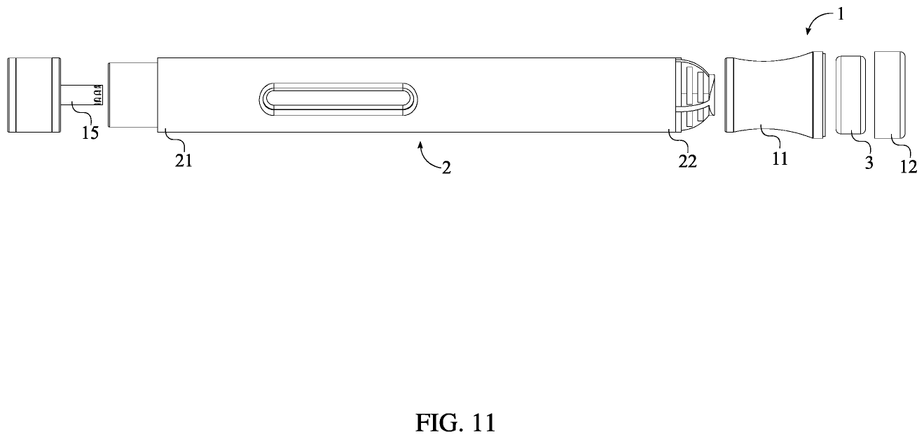

A needle shield fitted over the needle of the body 2 prevents the needle from being contaminated and/or damaged prior to the delivery of the injection. To remove the needle shield, the multi-function cap assembly 1 further comprises a needle-shield remover 15. The body 2 houses the needle as the body 2 is an elongated body, and the elongated body comprises a first end 21 and a second end 22. As such, the coupling interface 14 is engaged to the first end 21. More specifically, the needle-shield remover 15 is a slender tube that fits over the needle shield. Further, the needle-shield remover 15 is mounted into the receptacle 11, wherein the needle-shield remover 15 covers a needle of the autoinjector housing when the multi-function cap assembly 1 is connected to the body 2. As such, when the multi-function cap assembly 1 is pulled off the body 2, the needle-shield remover 15 pulls the needle shield off the needle. Referring to FIG. 10 and FIG. 11, in an alternate embodiment of the present invention, the coupling interface 14 is engaged to the second end 22, and the needle-shield remover 15 is mounted onto the first end 21. This arrangement allows the needle within the body 2 to remain covered with the needle-shield remover 15 while engaging with the antiseptic-soaked dressing 3 housed within the receptacle 11 and the removable cover 12.

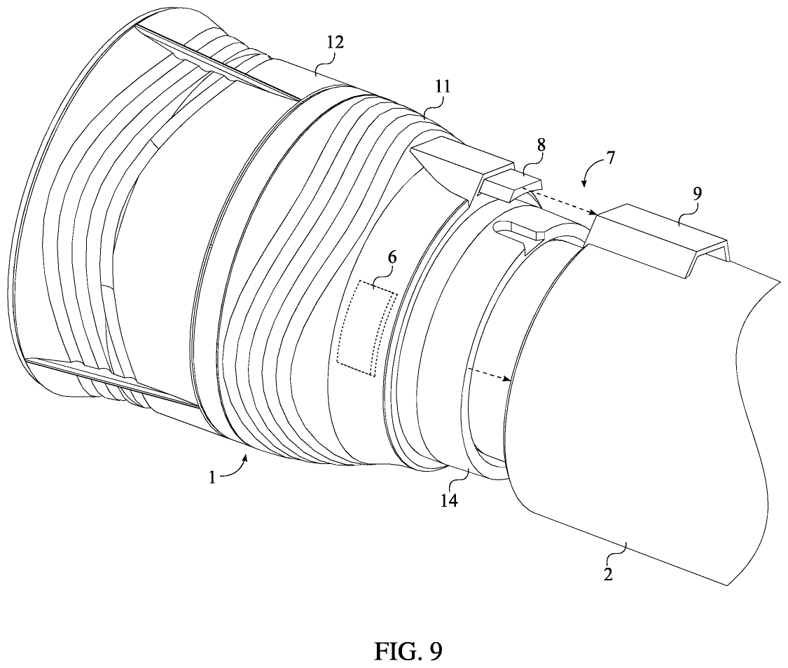

Referring to FIG. 9, preferably, the body 2 is used to inject liquid or gas solutions with limited shelf-life that are ineffective past the expiration date. Thus, the present invention comprises an expiration timer 6, which keeps track of the expiration date, and a locking mechanism 7, which prevents the delivery of expired solutions. The expiration timer 6 is mounted within the multi-function cap assembly 1 and can engage the locking mechanism 7 once the expiration date has passed. Moreover, the locking mechanism 7 is operatively integrated into the multi-function cap assembly 1, wherein the locking mechanism 7 is used to selectively secure the multi-function cap assembly 1 to the body 2. As such, the locking mechanism 7 creates a permanent lock between the multi-function cap assembly 1 and the body 2, thereby sealing the needle in the body 2. Similarly, the expiration timer 6 is operatively coupled to the locking mechanism 7, wherein the expiration timer 6 is used to actuate the locking mechanism 7. Preferably, the expiration timer 6 is a microcontroller programmed to keep track of the expiration date. In one possible embodiment, the expiration timer 6 may be programmed during the fabrication of the multi-function cap assembly 1. Alternately, a user interface may be provided to allow the administrator to program the expiration timer 6.

Since the multi-function cap assembly 1 is designed to be retrofitted onto existing autoinjectors, it is desirable to integrate both the expiration timer 6 and the locking mechanism 7 into the multi-function cap assembly 1. Accordingly, the locking mechanism 7 is integrated into the removable cover 12. More specifically, the locking mechanism 7 comprises a male connector 8 and the female connector 9. The female connector 9 is laterally mounted onto the body 2. The male connector 8 is slidably mounted onto the removable cover 12. A longitudinal axis of the male connector 8 is coincident to a longitudinal axis of the female connector 9. In the preferred embodiment of the present invention, the male connector 8 and the female connector 9 may operate similarly to a snap-fit buckle. The male connector 8 may comprise cantilever member, and the female connector 9 may comprise a slot. Once the expiration date passes, the expiration timer 6 activates the locking mechanism 7 by sliding the cantilever member into the slot. This forms a snap-fit between the male connector 8 and the female connector 9, which permanently secures the multi-function cap assembly 1 to the body 2.

Alternately, the locking mechanism 7 may comprise any number of fastening mechanisms well known in the arts. For example, the locking mechanism 7 may comprise screws, snaps, magnets, or any number of fasteners well known in the arts.

Although the invention has been explained in relation to its preferred embodiment, it is to be understood that many other possible modifications and variations can be made without departing from the spirit and scope of the invention as hereinafter claimed.

What is claimed is:

1. A system for integrating an antibacterial-element receptacle into a multi-function cap, wherein the system comprises:
a multi-function cap assembly;
a body;
an antiseptic-soaked dressing;
the multi-function cap assembly comprises a receptacle, a removable cover, a cover fastener, and a coupling interface;
the removable cover being attached across an opening of the receptacle by the cover fastener;
the coupling interface being connected adjacent to the receptacle, opposite the removable cover;
the coupling interface being engaged to the body; and,
the antiseptic-soaked dressing being mounted in between the removable cover and the receptacle.

2. The system as claimed in claim 1, wherein the coupling interface is a slip-fit fastener.

3. The system as claimed in claim 1, wherein the system further comprises:
the antiseptic-soaked dressing being connected into the receptacle; and
the antiseptic-soaked dressing traversing out of the opening of the receptacle.

4. The system as claimed in claim 1, wherein the system further comprises:
the antiseptic-soaked dressing being connected onto the removable cover; and
the antiseptic-soaked dressing traversing into the opening of the receptacle.

5. The system as claimed in claim 4, wherein the system further comprises:
a storage compartment;
a cover lid;
the storage compartment being integrated into the removable cover, opposite to the receptacle; and
the cover lid being attached across an opening of the storage compartment.

6. The system as claimed in claim 4, wherein the system further comprises:
the removable cover being threadably engaged to the receptacle.

7. The system as claimed in claim 1, wherein the system further comprises:
the multi-function cap assembly further comprises a needle-shield remover;
the body being an elongated body;
the elongated body comprises a first end and a second end;
the coupling interface being engaged to the first end; and,
the needle-shield remover being mounted into the receptacle.

8. The system as claimed in claim 1, wherein the system further comprises:
the multi-function cap assembly further comprises a needle-shield remover;
the body being an elongated body;
the elongated body comprises a first end and a second end;
the coupling interface being engaged to the second end; and,
the needle-shield remover being mounted onto the first end.

9. The system as claimed in claim 1, wherein the system further comprises:
an expiration timer;
a locking mechanism;
the expiration timer being mounted within the multi-function cap assembly;
the locking mechanism being operatively integrated into the multi-function cap assembly, wherein the locking mechanism is used to selectively secure the multi-function cap assembly to the body; and
the expiration timer being operatively coupled to the locking mechanism, wherein the expiration timer is used to actuate the locking mechanism.

10. The system as claimed in claim 9, wherein the system further comprises:
the locking mechanism comprises a male connector and a female connector;
the female connector being laterally mounted onto the body;
the male connector being slidably mounted onto the removable cover; and,
a longitudinal axis of the male connector being coincident to a longitudinal axis of the female connector.
